(19) United States
(12) Patent Application Publication
(10) Pub. No.: US 2022/0351114 A1
(43) Pub. Date: Nov. 3, 2022

(54) INFORMATION COLLECTING APPARATUS AND METHOD

(71) Applicant: NIPPON TELEGRAPH AND TELEPHONE CORPORATION, Tokyo (JP)

(72) Inventors: Rie SAKAI, Musashino-shi, Tokyo (JP); Hiroshi YOSHIDA, Musashino-shi, Tokyo (JP); Naoki NISHIYAMA, Musashino-shi, Tokyo (JP); Tomoko SHIBATA, Musashino-shi, Tokyo (JP)

(73) Assignee: NIPPON TELEGRAPH AND TELEPHONE CORPORATION, Tokyo (JP)

(21) Appl. No.: 17/634,632

(22) PCT Filed: Aug. 20, 2019
(86) PCT No.: PCT/JP2019/032508

§ 371 Date: Feb. 11, 2022

(51) Int. Cl. G06Q 10/06; G06Q 50/26

(52) U.S. Cl. CPC G06Q 10/063114; G06Q 50/265; A61B 5/1117

(57) ABSTRACT

An information collecting apparatus according to an embodiment includes a sensor data acquisition unit configured to acquire sensor data related to work performed by a worker, a work information data generation unit configured to generate work information data related to work performed by the worker for a specific period of time based on the sensor data acquired by the sensor data acquisition unit, and a danger information specification unit configured to specify work information data related to an action that indicates danger as dangerous work information data indicating dangerous work performed by the worker when an action of the worker in a predetermined period of time, which is indicated by the work information data generated, is the action that indicates danger.

### TECHNICAL FIELD

An embodiment of the present invention relates to an information collecting apparatus and a method.

### BACKGROUND ART

In construction work such as telecommunications construction work, the occurrence of workers' accidents resulting in injury or death has become a problem. Many accidents resulting in injury or death are related to, for example, falls during the work performed by workers.

### CITATION LIST

### Patent Literature

PTL 1: JP 2010-117938 A

### SUMMARY OF THE INVENTION

### Technical Problem

Among the construction work, especially telecommunications construction work and the like have many processes in which a worker is required to work alone. In construction work, for example, in a case such as (1) work that did not lead to an accident resulting in injury or death but is one step before the accident, a so-called potential accident or (2) an accident that occurred but that can be solved by a worker on his/her own without going to a hospital or the like, the potential accident or accident mentioned above often fails to be reported to a manager due to embarrassment, guilt, or the like of disclosing an accident or work that may directly lead to an accident to others.

In addition, when a manager individually tries to ask each worker about an accident or the like, the worker may conceal the accident, and thus a potential accident or an accident could not be objectively or systematically extracted.

The present invention is made in view of the circumstances described above, and an object is to provide an information collecting apparatus and a method which make it possible to acquire information indicating danger in work without requiring a worker's reporting.

### Means for Solving the Problem

An information collecting apparatus according to an aspect of the present invention includes a sensor data acquisition unit configured to acquire sensor data related to work performed by a worker, a work information data generation unit configured to generate work information data related to work performed by the worker for a specific period of time based on the sensor data acquired by the sensor data acquisition unit, and a danger information specification unit configured to specify work information data related to an action that indicates danger as dangerous work information data indicating dangerous work performed by the worker when an action of the worker in a predetermined period of time, which is indicated by the work information data generated, is the action that indicates danger.

An information collecting method according to an aspect of the present invention is a method performed by an information collecting apparatus, and includes acquiring sensor data related to work performed by a worker, generating work information data related to work performed by the worker for a specific period of time based on the sensor data acquired by the sensor data acquisition unit; and when an action of the worker in a predetermined period of time, which is indicated by work information data generated, is an action that indicates danger, specifying work information data related to the action that indicates danger as dangerous work information data indicating dangerous work performed by the worker.

### Effects of the Invention

According to the present invention, information indicating danger in work can be acquired without requiring a worker's reporting.

### BRIEF DESCRIPTION OF DRAWINGS

FIG. 1 is a diagram illustrating an example of an overall configuration of an information collecting apparatus according to an embodiment of the present invention.

FIG. 2 is a block diagram illustrating an example of a hardware configuration of the information collecting apparatus according to the embodiment of the present invention.

FIG. 3 is a block diagram illustrating an example of a software configuration of the information collecting apparatus according to the embodiment of the present invention.

FIG. 4 is a flowchart illustrating an example of a processing operation of the information collecting apparatus according to the embodiment of the present invention.

FIG. 5 is a diagram illustrating an example of detection of acceleration and height.

FIG. 6 is a diagram illustrating an example of dangerous work information data related to work information data.

FIG. 7 is a diagram illustrating an example of acceleration related to dangerous work information data.

FIG. 8 is a diagram illustrating an example of the voice volume related to dangerous work information data.

FIG. 9 is a diagram illustrating an example of an output of unreported dangerous work information data.

FIG. 10 is a diagram illustrating a display example of a dangerous work report.

### DESCRIPTION OF EMBODIMENTS

Hereinafter, an embodiment according to the present invention will be described with reference to drawings.

### Configuration

### (1) System

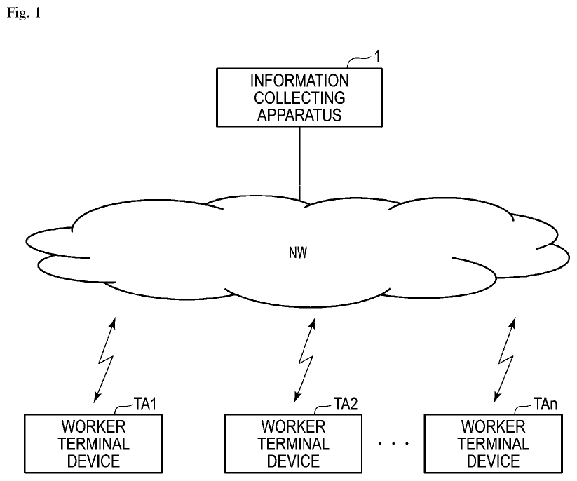

FIG. 1 is a diagram illustrating an example of an overall configuration of a system including an information collecting apparatus according to an embodiment of the present invention.

The system can provide the information collecting apparatus 1 on, for example, the Web or the cloud. Further, the information collecting apparatus 1 can communicate with a plurality of worker terminal devices TA1, TA2, . . . , and TAn having a communication function via a network NW. Note that the plurality of worker terminal devices may be representatively referred to as a “worker terminal device TA” or a “worker terminal”. Here, it is assumed that one worker terminal device is always carried by each worker.

The network NW is composed of, for example, a relay network and a plurality of access networks for accessing the relay network. The relay network used is a public network such as the general Internet or a closed network that is controlled to be accessed only from a limited device, or the like. Examples of the access network used include a wireless local area network (LAN), a mobile phone network, a wired telephone network.

### (2) Information Collecting Apparatus

### (2-1) Hardware Configuration

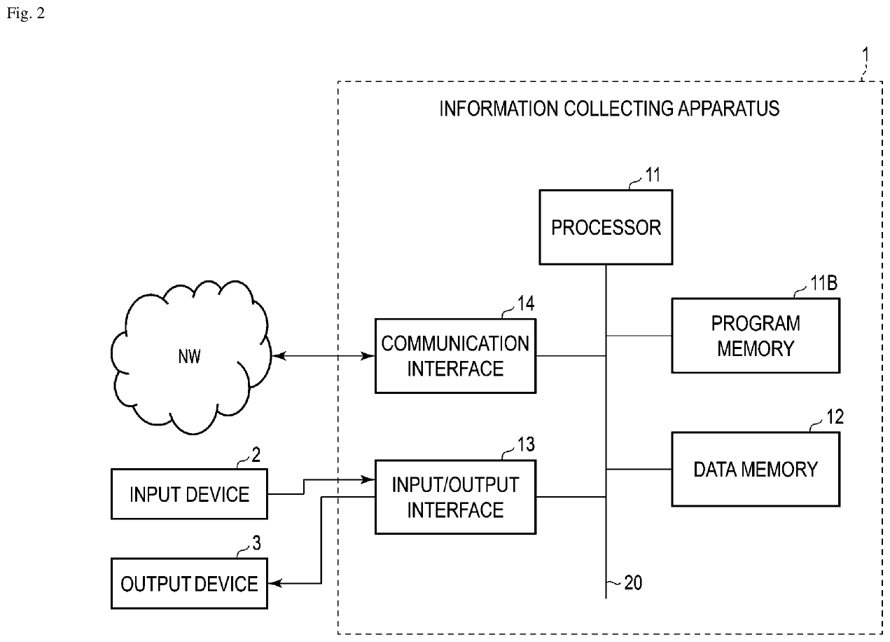

FIG. 2 is a block diagram illustrating an example of a hardware configuration of the information collecting apparatus 1 according to the embodiment of the present invention. The information collecting apparatus 1 is composed of, for example, a server computer or a personal computer, and includes a hardware processor 11A such as a central processing unit (CPU). Further, a program memory 11B, a data memory 12, an input/output interface 13, and a communication interface 14 are connected to the hardware processor 11A via a bus 20.

The communication interface 14 includes, for example, one or more wireless communication interface units to allow transmission/reception of information to/from external equipment including the worker terminal device TA. As a wireless interface, for example, an interface with a small power wireless data communication standard adopted such as a wireless LAN is used.

An input device 2 and an output device 3 for a manager, which are attached to the information collecting apparatus 1, are connected to the input/output interface 13. The input/output interface 13 performs processing for taking in operation data input by a manager through the input device 2 such as a keyboard, a touch panel, a touch pad, or a mouse and outputting output data to the output device 3 including a display device using liquid crystal, organic electro luminescence (EL), or the like and a speaker outputting voice, to display the output data. Note that, as the input device 2 and the output device 3, a device built into the information collecting apparatus 1 may be used, and an input device and an output device of another information terminal capable of performing communication through the network NW may also be used.

The program memory 11B is a memory, for example, in which a non-volatile memory such as a hard disk drive (HDD) or a solid state drive (SSD) allowing writing and reading at any time and a non-volatile memory such as a read only memory (ROM) are used in combination as a non-temporary tangible storage medium, in which a program required to execute various types of control processing according to the embodiment is stored.

The data memory 12 is a memory in which, for example, a non-volatile memory allowing writing and reading at any time such as an HDD or an SSD and a volatile memory such as a random access memory (RAM) are used in combination as a tangible storage medium, and is used to store various data acquired and created in the course of performing information collecting processing.

### (2-2) Software Configuration

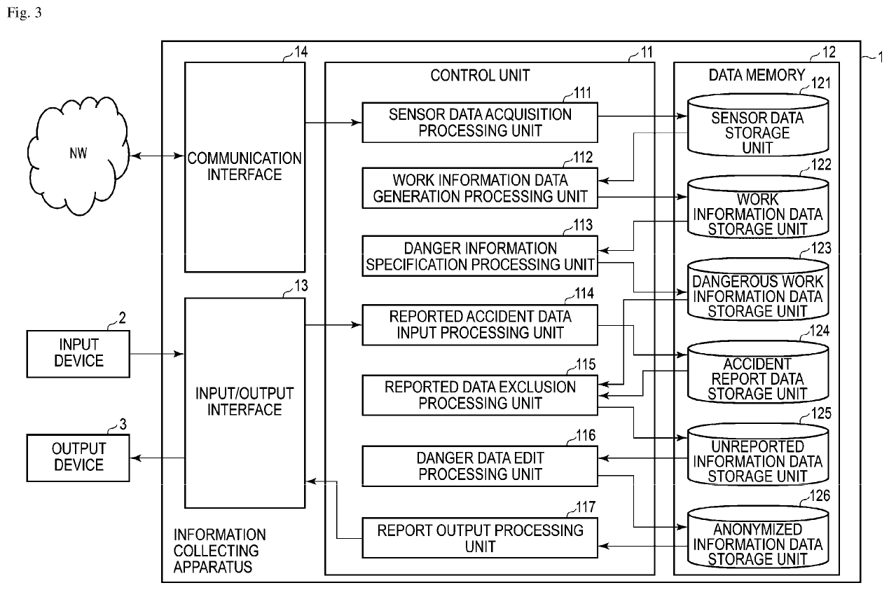

FIG. 3 is a block diagram illustrating an example of a software configuration of the information collecting apparatus 1 according to the embodiment of the present invention. In FIG. 3, the software configuration of the information collecting apparatus 1 is illustrated in association with the hardware configuration illustrated in FIG. 2.

The storage region of the data memory 12 is provided with a sensor data storage unit 121, a work information data storage unit 122, a dangerous work information data storage unit 123, an accident report data storage unit 124, an unreported information data storage unit 125, and an anonymized information data storage unit 126.

However, the storage units 121 to 126 are not essential configurations in the information collecting apparatus 1, and may be provided in an external storage medium, such as a universal serial bus (USB) memory or a storage device, such as a database server disposed in the cloud.

The control unit 11 is composed of the hardware processor 11A and the program memory 11B, and includes a sensor data acquisition processing unit 111, a work information data generation processing unit 112, a danger information specification processing unit 113, an reported accident data input processing unit 114, a reported data exclusion processing unit 115, a danger data edit processing unit 116, and a report output processing unit 117 as processing functional units by software.

All of the processing functions of these units are achieved by causing the hardware processor 11A to execute a program stored in the program memory 11B. Note that these processing functions may be achieved not by using a program stored in the program memory 11B, but by using a program provided through a network. The control unit 11 may be achieved in various other formats including an integrated circuit such as an application specific integrated circuit (ASIC) or a field-programmable gate array (FPGA).

Operation

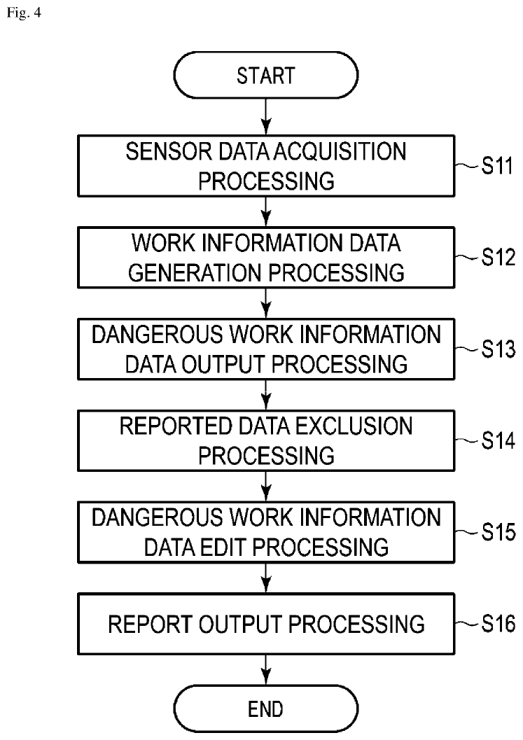

FIG. 4 is a flowchart illustrating an example of processing operations of the information collecting apparatus 1 according to the embodiment of the present invention.

The sensor data acquisition processing unit 111 acquires sensor data which is related to a worker and transmitted from the worker terminal device TA through the communication interface 14, and identification information for identifying a worker who owns the worker terminal device TA from the worker terminal device TA.

The sensor data acquisition processing unit 111 performs processing for storing the acquired results in the sensor data storage unit 121 together with the current time (S11). The current time is kept by the sensor data acquisition processing unit 111 or the worker terminal device TA.

Here, the sensor data may include, for example, (1) acceleration according to the movement of a worker, (2) a height at which the worker is located, (3) voice uttered by the worker, (4) a video of the worker, (5) positional information of the worker, and (6) a centroid oscillation area of the worker. It is assumed that the sensor data is sequentially acquired at predetermined time intervals with sensor data, separated for a predetermined period of time, for example, for 10 seconds from a predetermined timing, which is continuous in time series as one unit.

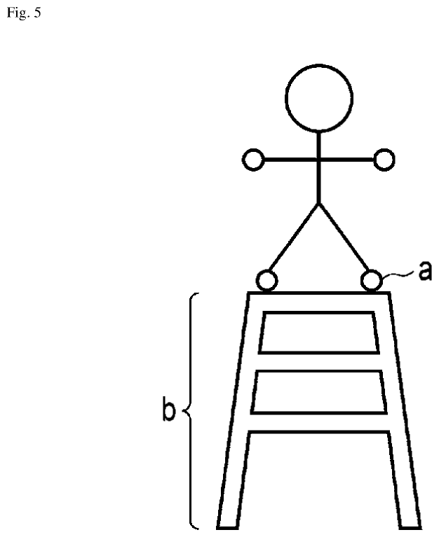

FIG. 5 is a diagram illustrating an example of detection of acceleration and height. As illustrated in FIG. 5, acceleration related to the movement of a worker may be detected by an acceleration sensor capable of being worn on the worker's foot (a in FIG. 5), wrist, or the like, and may be transmitted from the worker terminal device TA to the information collecting apparatus 1. The acceleration sensor may be built into the worker terminal device TA. The height (b in FIG. 5) and positional information of the worker may be detected by, for example, a global positioning system (GPS) sensor built into the worker terminal device TA and may be transmitted from the worker terminal device TA to the information collecting apparatus 1.

The GPS sensor receives a GPS signal transmitted from a plurality of GPS satellites and performs a ranging operation to calculate the latitude, longitude, and altitude of the worker, and GPS data including the calculated latitude, longitude, and the like is transmitted to the information collecting apparatus 1. Note that the acquisition of positional information is not limited to a method based on signals received from the GPS satellites, and other methods such as using positional information of a wireless base station or a Wi-Fi access point may be used as long as the same function is exhibited.

Regarding the centroid oscillation area described above, data indicating the centroid oscillation area measured by, for example, a stabilometer may be transmitted to the worker terminal device TA and transmitted to the information collecting apparatus 1 via the worker terminal device TA.

Voice uttered by the worker may be detected by, for example, a microphone built into the worker terminal device TA and may be transmitted from the worker terminal device TA to the information collecting apparatus 1 as voice data.

A video of the worker may be captured by, for example, a camera built into the worker terminal device TA and may be transmitted from the worker terminal device TA to the information collecting apparatus 1 as video data.

As the camera, for example, a solid-state imaging device such as a charge coupled device (CCD) or a complementary metal oxide semiconductor (CMOS) sensor may be used. Furthermore, the camera may be an infrared camera. In addition, data acquired by the camera may be moving image (video) data or still image data captured at fixed time intervals.

The work information data generation processing unit 112 reads sensor data and identification information stored in the sensor data storage unit 121 in S11.

In the data memory 12 of the information collecting apparatus 1, personal information of the worker, for example, the name, is stored in association with the identification information of the worker.
The work information data generation processing unit 112 performs processing for reading the personal information associated with the identification information read from the sensor data storage unit 121 from the data memory 12, generating work information data in which the personal information and the read sensor data are associated with each other, and storing the work information data in the work information data storage unit 122 (S12).

Next, the danger information specification processing unit 113 reads work information data regarding a certain worker and stored in the work information data storage unit 122 in S12, and aggregates each of (1) the number of times acceleration in the work information data exceeds a threshold value, (2) the number of times height in the work information data exceeds a threshold value, (3) the number of times the voice volume indicated by voice in the work information data exceeds a threshold value, and (4) the number of keywords indicating the danger of the worker included in the voice in the work information data, during the same period of time for the work information data in certain units.

Keywords indicating the danger of the worker are, for example, “dangerous,” “fall,” and the like.

When, for example, the following condition A is satisfied and at least one of the following condition B or C is satisfied based on results of the aggregation mentioned above, the danger information specification processing unit 113 regards the work information data to be aggregated as data indicating dangerous work performed by a worker related to the work information data, extracts the work information data to be aggregated as dangerous work information data, and stores, in the dangerous work information data storage unit 123, the work information data extracted, together with work information data which is not dangerous work information data for the same worker (S13).

Condition A: The number of times acceleration in the work information data exceeds a threshold value is once or more, and the number of times height in the work information data exceeds a threshold value is once or more. The threshold value related to the height is, for example, a value obtained in verification of a fall.
Condition B: The number of times the voice volume indicated by voice in the work information data exceeds a threshold value is once or more. The threshold value related to the voice volume is, for example, a normal voice volume.
Condition C: The voice in the work information data includes one or more predetermined keywords indicating the danger of the worker.

Processing for data related to the same period of time, the same position, and the same worker will be described below. In addition, data handled by the reported accident data input processing unit 114, the reported data exclusion processing unit 115, the danger data edit processing unit 116, and the report output processing unit 117 of the control unit 11 includes work information data which is not dangerous work information data related to the same period of time, the same position, and the same worker.

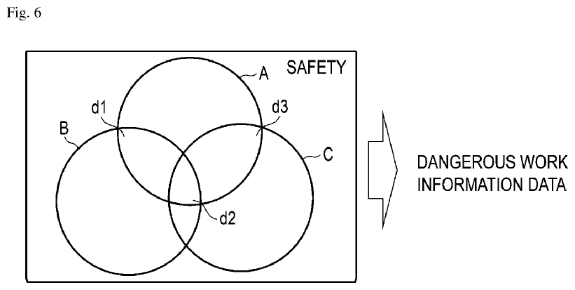

FIG. 6 is a diagram illustrating an example of dangerous work information data related to work information data.

The range A in work information data surrounded by a frame illustrated in FIG. 6 indicates the range of work information data satisfying the condition A described above among the work information data. The range B in the work information data surrounded by a frame illustrated in FIG. 6 indicates the range of work information data satisfying the condition B described above among the work information data. The range C in the work information data surrounded by a frame illustrated in FIG. 6 indicates a working information range of data satisfying the condition C described above among the work information data.

The d1 illustrated in FIG. 6 indicates the range of dangerous work data satisfying both of the conditions A and B described above among the work information data surrounded by the frame illustrated in FIG. 6. The d2 illustrated in FIG. 6 indicates the range of dangerous work data satisfying all of the conditions A, B, and C described above among the work information data surrounded by the frame illustrated in FIG. 6. The d3 illustrated in FIG. 6 indicates the range of dangerous work data satisfying both of the conditions A and C described above among the work information data surrounded by the frame illustrated in FIG. 6. Furthermore, the other ranges in the work information data surrounded by the frame illustrated in FIG. 6 are ranges corresponding to safe work that is not dangerous.

That is, the information collecting apparatus 1 according to the present embodiment can specify dangerous work performed by a worker using acceleration, height, the volume of voice, and the number of keywords included in the voice volume which are detected during work performed by the worker, and can specify a potential accident or an unreported accident without requiring a worker's reporting.

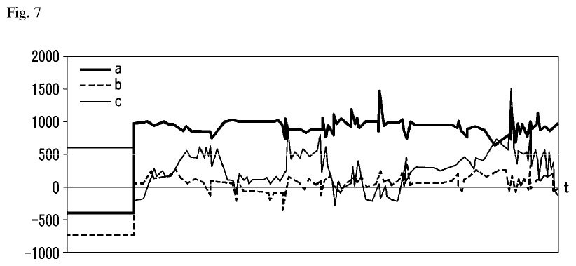

FIG. 7 is a diagram illustrating an example of acceleration related to dangerous work information data.

In the example illustrated in FIG. 7, accelerations a, b, and c obtained when a worker is working on a stepladder are illustrated. In the example illustrated in FIG. 7, the value of the acceleration a may exceed a threshold value of 1000. Here, a case where the value of acceleration exceeds a threshold value of 1000 like the acceleration a indicates a state where the worker falls from the stepladder or is highly likely to fall from the stepladder.

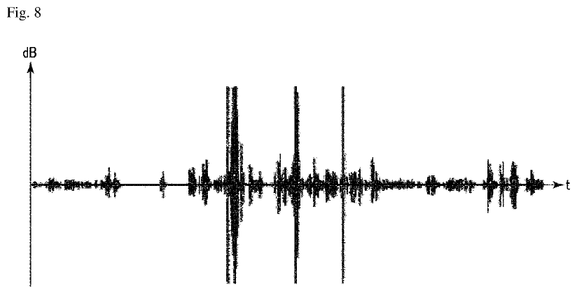

FIG. 8 is a diagram illustrating an example of the voice volume related to dangerous work information data.

The vertical axis illustrated in FIG. 8 represents the degree of the voice volume. Here, the example indicates a state where a worker falls from a stepladder or is highly likely to fall from the stepladder when the degree of the voice volume is greater than in a normal case.

Here, for example, the input device 2 receives a manager's input operation of positional information, a worker name, and occurrence time of an accident which is related to an accident resulting in injury or death indicated in a past accident report, which has already occurred and has been reported to the manager by the worker.

The reported accident data input processing unit 114 inputs positional information, a worker name, and occurrence time of an accident of the worker which is related to an accident resulting in injury or death indicated in the past accident report via the input/output interface 13. The reported accident data input processing unit 114 performs processing for sequentially storing the input results in the accident report data storage unit 124 as reported accident data.

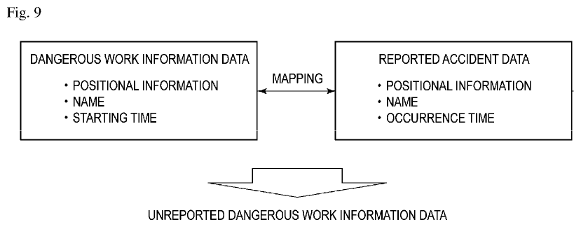

FIG. 9 is a diagram illustrating an example of output of unreported dangerous work information data.

As illustrated in FIG. 9, the reported data exclusion processing unit 115 collects positional information, a worker name, and occurrence time of an accident of a worker which is related to an accident resulting in injury or death indicated in an accident report from the reported accident data stored in the accident report data storage unit 124.

Then, the reported data exclusion processing unit 115 performs mapping of a combination of positional information, a name, and occurrence time collected from the accident report data storage unit 124 and a combination of positional information, a name, and occurrence time of dangerous work information data stored in the dangerous work information data storage unit 123 in S13.

By the mapping, the reported data exclusion processing unit 115 generates data in which data including a combination of positional information, a worker name, and occurrence time matching a combination of positional information, a worker name, and occurrence time included in reported accident data is excluded from the dangerous work information data stored in the dangerous work information data storage unit 123.

The reported data exclusion processing unit 115 stores the data after the exclusion in the unreported information data storage unit 125 as unreported dangerous work information data (S14). In this manner, data in which a position, a name, and occurrence time overlap those in reported accident data is excluded from the dangerous work information data stored in the dangerous work information data storage unit 123 in S13, and thus the dangerous work information data stored in the dangerous work information data storage unit 123 in S13 can be narrowed down to data that has not yet been reported to a manager.

The danger data edit processing unit 116 reads the unreported dangerous work information data stored in the unreported information data storage unit 125 in S14 and generates anonymized unreported dangerous work information data by (1) anonymizing a worker name, (2) eliminating face information of the worker from video information, and (3) changing a voice in voice information with respect to the unreported dangerous work information data.

To be specific, to anonymize the unreported dangerous work information data, the danger data edit processing unit 116 converts the name of a person included in conversation content of voice information into an anonymous name and also converts a voice tone into a mechanical voice tone.

In addition, the danger data edit processing unit 116 replaces the face of a person in video information with a smile mark or the like.

The danger data edit processing unit 116 stores the generated anonymized unreported dangerous work information data in the anonymized information data storage unit 126 (S15). Performing anonymization processing on the unreported dangerous work information data in this manner can prevent a worker involved in dangerous work from being identified by a manager. The report output processing unit 117 generates a dangerous work report for visualizing dangerous work and showing it to a manager based on the anonymized unreported dangerous work information data stored in the anonymized information data storage unit 126 in S15, and outputs the dangerous work report to the output device 3 via the input/output interface 13 (S16).

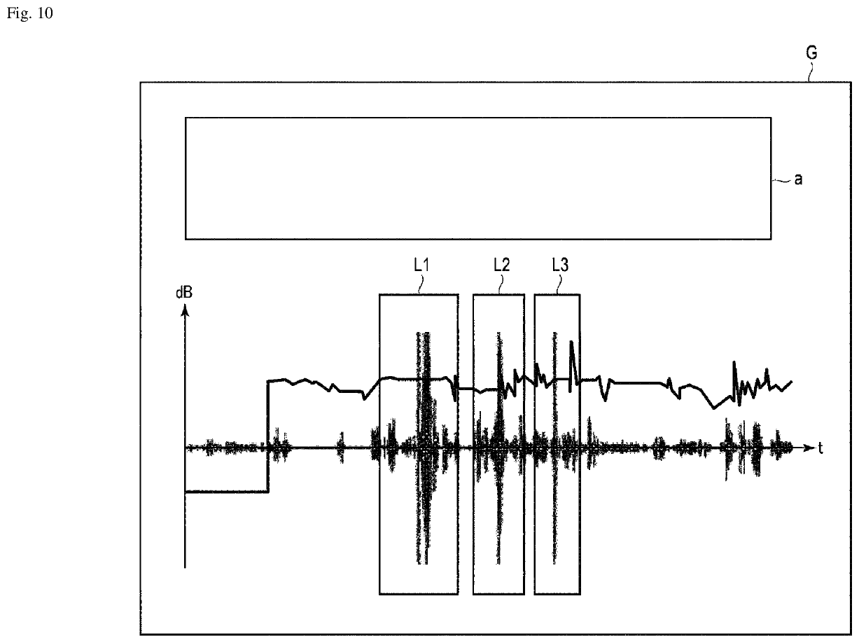

FIG. 10 is a diagram illustrating a display example of a dangerous work report. Here, it is assumed that the output device 3 is a display and a display screen G of the dangerous work report illustrated in FIG. 10 is displayed on the display. In the example illustrated in FIG. 10, the dangerous work report indicates video information (a illustrated in FIG. 10). In a dangerous work report, graphs of acceleration and the voice volume within a fixed period of time in which acceleration and the voice volume in dangerous work data are present, among graphs of acceleration and the volume of voice in work information data during a certain period of time, are surrounded by frames L1, L2, and L3 and distinguished from each other. This enables a manager to visualize timing at which dangerous work exists.

In addition, the dangerous work report can also include a centroid oscillation area indicated by the work information data in the period of time described above, the number of keywords included in voice, height information of a worker, and the like.

As described above, the information collecting apparatus according to the present embodiment acquires sensor data related to work performed by a worker and generates work information data related to work performed by a worker for a specific period of time based on the sensor data. The information collecting apparatus specifies dangerous work information data indicating a worker's dangerous work when the worker's action indicated by the work information data is an action that indicates danger. Thus, the information collecting apparatus can acquire information indicating danger in work without requiring a worker's reporting.

Further, a scheme described in each of the embodiments is stored in a recording medium such as a magnetic disk (a Floppy (trade name) disk, a hard disk, or the like), an optical disc (a CD-ROM, a DVD, an MO, or the like), a semiconductor memory (a ROM, a RAM, a flash memory, or the like) or transferred by a communication medium for distribution, as a program (a software unit) that can be executed by a calculator (a computer). The program stored in the medium also includes a setting program incorporating a software unit (including not only an execution program but also a table or data structure), which will be executed in a calculator, into the calculator. A calculator actualizing the present apparatus executes the process described above by loading the program recorded on the recording medium or constructing a software unit using the setting program in some cases, and controlling an operation using the software unit. Note that the recording medium referred to herein is not limited to a recording medium for distribution but includes a storage medium such as a magnetic disk or a semiconductor memory provided in the calculator or a device connected thereto via a network.

It is to be noted that the present invention is not limited to the embodiments mentioned above and can be variously modified in the implementation stage without departing from the gist of the present invention. In addition, an appropriate combination of the embodiments can also be implemented, in which a combination of their effects can be obtained. Further, the above embodiments include various inventions, which can be designed by combining constituent elements selected from a plurality of constituent elements disclosed here. For example, a configuration in which some constituent elements are removed from all the constituent elements illustrated in the embodiments can be designed as an invention if the problems can be solved and the effects can be achieved.

### REFERENCE SIGNS LIST

1 Information collecting apparatus
2 Input device
3 Output device
11 Control unit
11A Hardware processor
11B Program memory
12 Data memory
13 Input/output interface
14 Communication interface
20 Bus
111 Sensor data acquisition processing unit
112 Work information data generation processing unit
113 Danger information specification processing unit
114 Accident data input processing unit
115 Data exclusion processing unit
116 Danger data edit processing unit
117 Report output processing unit
121 Sensor data storage unit
122 Work information data storage unit
123 Dangerous work information data storage unit
124 Accident report data storage unit
125 Unreported information data storage unit
126 Anonymized information data storage unit

1. An information collecting apparatus comprising:
a processor; and
a storage medium having computer program instructions stored thereon, when executed by the processor, perform to:
acquire sensor data related to work performed by a worker;
generate work information data related to work performed by the worker for a specific period of time based on the sensor data acquired by the sensor data acquisition unit; and
specify work information data related to an action that indicates danger as dangerous work information data indicating dangerous work performed by the worker when an action of the worker in a predetermined period of time, which is indicated by the work information data generated, is the action that indicates danger.

2. The information collecting apparatus according to claim 1, wherein the computer program instructions further perform to specifies the work information data related to the action that indicates danger as the dangerous work information data in a case where it is determined that the action of the worker is an action that indicates danger based on a combination condition of any two or more of the number of times acceleration in the work information data exceeds a threshold value for a predetermined period of time, the number of times height in the work information data exceeds a threshold value for the predetermined period of time, the number of times voice volume indicated by voice in the work information data exceeds a threshold value for the predetermined period of time, and the number of keywords indicating danger of the worker included in the voice in the work information data for the predetermined period of time.

3. The information collecting apparatus according to claim 1, wherein
the dangerous work information data includes positional information of a worker, a worker name, and occurrence time of work, and
the information collecting apparatus further comprises:
an accident data storage unit configured to store accident data including positional information related to an accident, a worker name, and occurrence time which were reported by a worker in the past, and
wherein the computer program instructions further perform to output unreported dangerous work information data in which data including positional information, a name, and occurrence time of work matching the positional information, the name, and the occurrence time of work included in the accident data stored in the accident data storage unit is excluded from the dangerous work information data.

4. The information collecting apparatus according to claim 3, wherein the computer program instructions further perform to generate anonymized unreported dangerous work information data obtained by performing anonymization processing on the unreported dangerous work information data output by the output unit; and
a report generation unit configured to generate a dangerous work report for visualizing the anonymized unreported dangerous work information data that is generated.

5. An information collecting method performed by an information collecting apparatus, the method comprising:
acquiring sensor data related to work performed by a worker;
generating work information data related to work performed by the worker for a specific period of time based on the sensor data acquired; and
when an action of the worker in a predetermined period of time, which is indicated by the work information data generated, is an action that indicates danger, specifying work information data related to the action that indicates danger as dangerous work information data indicating dangerous work performed by the worker.

6. The information collecting method according to claim 5, further comprising:
specifying the work information data related to the action that indicates danger as the dangerous work information data in a case where it is determined that the action of the worker is an action that indicates danger based on a combination condition of any two or more of the number of times acceleration in the work information data exceeds a threshold value for a predetermined period of time, the number of times height in the work information data exceeds a threshold value for the predetermined period of time, the number of times voice volume indicated by voice in the work information data exceeds a threshold value for the predetermined period of time, and the number of keywords indicating danger of the worker included in the voice in the work information data for the predetermined period of time.

7. The information collecting method according to claim 5, wherein
the dangerous work information data includes positional information of a worker, a worker name, and occurrence time of work, and
the information collecting method further comprises
outputting unreported dangerous work information data in which data including positional information, a name, and occurrence time of work matching the positional information, the name, and the occurrence time of work included in the accident data stored in an accident data storage unit, which stores accident data including positional information related to an accident, a worker name, and occurrence time which were reported by a worker in the past, is excluded from the dangerous work information data that is specified.
